(19) United States
(12) Patent Application Publication
(10) Pub. No.: US 2003/0099599 A1
(43) Pub. Date: May 29, 2003

(54) Antibiotic spray

(76) Inventor: Ora Shuval-Sudai, Jerusalem (IL)

Correspondence Address: Rashida A Karmali, Esq., 10th Floor, 99 Wall Street, New York, NY, 10005, US

(21) Appl. No.: 10/301,130

(22) Filed: Nov. 21, 2002

(30) Foreign Application Priority Data
Nov. 25, 2001 (IL) 146,725

(51) Int. Cl. A61K 31/43; A61L 9/04

(52) U.S. Cl. 424/43; 514/192

(57) ABSTRACT

The invention provides a propellant free, non-pressurized spray dispenser containing a suspension of an antibiotic effective for the treatment of group A streptococci for topical application to the throat.

The present invention relates to a spray dispenser and to the use of a spray of an antibiotic effective for the treatment of group A streptococcal (GAS) tonsillopharyngitis for prophylactic prevention and supplemental topical treatment of Streptococcal throat infections as well as for treatment of acute tonsillopharyngitis.

Streptococcal tonsillopharyngitis is a common infection in children and adolescents. The great majority of these infections are caused by group A beta-haemolytic streptococci (GABHS).

Group A streptococcal (GAS) tonsillopharyngitis has the potential for inducing serious non-purulent complications, such as rheumatic fever with rheumatic carditis and less frequently, glomerulonephritis.

Penicillin remains the antimicrobial agent of choice for the treatment of streptococcal tonsillopharyngitis, except in individuals with history of penicillin allergy in which case erythromycin is usually used. Other antimicrobial agents such as Azithromycin, clarithromycin, Cefalexn, cefadroxil, Cefuroxime and Clindamycin are less frequently used.

Streptococcal Resistance to penicillin has never been documented, however, nevertheless recurrences are a common finding after antibiotic treatment of acute group A streptococcal tonsillopharyngitis. About 25% of all GAS tonsillopharyngitis treated with penicillin recurs within 2 months. The clinical recurrence have resulted in medical as well as socioeconomic problems.

The reason for recurrent streptococcal throat infection, has been attributed to several factors including low patient compliance and low effective drug concentration at the site of infection (tonsilles), which in turn might be due to insufficient absorption of the antibiotic from the gastrointestinal tract.

The current recommendation in order to prevent recurrences, for children who suffer four or more episodes of GAS tonsillopharyngitis in a year, require daily continuous antibiotic prophylaxis for about 6 months utilizing about half of the normal penicillin dose used during an acute infection.

Tonsillectomy is indicated for children who have at 4-6 episodes of GAS tonsillopharyngitis in a year, and when the continuous antibiotic prophylaxis fails.

According to the present invention it has now been discovered that a prophylactic effect against the occurrence of infections caused by group A beta-haemolytic streptococci (GABHS) and a supplementary treatment of such infections can be achieved by the topical application of a propellant free, non-pressurized spray of a suspension containing an antibiotic effective for the treatment of group A streptoccocci directly to the throat area.

Thus, according to the present invention there is now provided a propellant free, non-pressurized spray dispenser containing a suspension of an antibiotic effective for the treatment of group A streptococci for topical application to the throat.

While said antibiotic can be any of the antibiotic suspensions mentioned above including penicillin V, Augmentin (amoxycillin and clavlanic acid) or erythromycin in preferred embodiments of the present invention said antibiotic is amoxycillin.

In another aspect of the present invention there is provided the use of a propellant free, non-pressurized suspension of an antibiotic effective for the treatment of group A streptococci for the manufacture of a spray dispenser of said suspension for topical application to the throat for prophylactic prevention and supplemental treatment of group A streptococcal (GAS) tonsillopharyngitis, as well as for treatment of acute tonsillopharyngitis.

The suspension of the antibiotic used in the present invention is the standard suspension produced in the pharmacy by mixing the powdered antibiotic with sterile water and thus the particle size of the powdered antibiotic is well above 100 microns.

It is known that with the outbreak of World War II the government increased its commitment and adopted a purposive medical research policy and that scientist from Coldspring Harbor Institute and Carnegie researchers used X-ray mutagenesis to produce a high-yielding strain of Penicillium mold that increased wartime penicillin production fivefold. Researchers at the Biological Laboratory developed the “Cold Spring Harbor Aeroliser” which produced a spray of super-fine penicillin droplets and biological laboratory scientist Vernon Bryson developed an aerosol penicillin spray for respiratory infections.

Similarly, U.S. Pat. No. 3,560,607 teaches and describes Aerosol formulations of finely divided solid medicaments with anionic surface-active agents which powdered medicament formulations for use in pressurized dispensing containers where designed for delivering pharmaceutical compositions to be ingested, e.g., inhaled, by the user.

Nevertheless, said technology from over 50 and 30 years ago, respectively, was directed to aerosol formulations for inhalation in to the respiratory tract and not to topical sprays adapted to be applied to the throat area for prophylactic prevention and supplemental treatment of group A streptococcal (GAS) tonsillopharyngitis.

Thus, despite the existence of said publications for several decades heretofore it has not been suggested to use local antibiotic treatment rather than systemic treatment for the prevention of recurrent GAS tonsillopharyngitis.

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of the preparation as well as of the principles and conceptual aspects of the invention.

### EXAMPLE 1

A plastic spray bottle with penicillin (amoxycillin) suspension 250 mg\5 cc was prepared. Every 5 cc supplies 70 puffs while every spray puff delivered 3.57 mg of penicillin (amoxycillin). The suspension can be administered by the patient himself as a spray toward his throat, 1 puff at each occasion, twice or more daily, for six months starting immediately after throat culture is taken. The spray bottle can be stored by the patient at +4 to +8 Celsius and is replaced every 10 days.

Children ages 5 and 8 placed on the spray regimen according to example 1 between the months of November 2000 and May 2001 did not develop any cases of group A streptococcal (GAS) tonsillopharyngitis, while the same children developed several cases thereof during the previous year when not placed on this regimen.

Since one of the reasons for penicillin failure, and relapse of GAS tonsillopharyngitis, is low antibiotic levels at the site of infection (0.67±0.3 microgr\for gr of tonsil tissue), the local spray treatment can be also considered and used as a supplement to antibiotic p.o treatment during an acute streptococcal throat infection.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

What is claimed is:

1. A propellant free, non-pressurized spray dispenser containing a suspension of an antibiotic effective for the treatment of group A streptococci for topical application to the throat.

2. A propellant free, non-pressurized spray dispenser, according to claim 1 wherein said antibiotic is a penicillin.

3. A propellant free, non-pressurized spray dispenser according to claim 2 wherein said penicillin is amoxycillin.

4. Use of a propellant free, non-pressurized suspension of an antibiotic effective for the treatment of group A streptococci for the manufacture of a spray dispenser of said suspension for topical application to the throat for prophylactic prevention and supplemental treatment of group A streptococcal (GAS) tonsillopharyngitis, as well as for treatment of acute tonsillopharyngitis.
